(19) United States
(12) Patent Application Publication
(10) Pub. No.: US 2015/0080702 A1
(43) Pub. Date: Mar. 19, 2015

(54) GENERATING COLONOSCOPY RECOMMENDATIONS

(71) Applicant: Mayo Foundation for Medical Education and Research, Rochester, MN (US)

(72) Inventors: Lisa A. Boardman, Rochester, MN (US); Kavishwar B. Wagholikar, Rochester, MN (US); Rajeev Chaudhry, Rochester, MN (US)

(21) Appl. No.: 14/482,938

(22) Filed: Sep. 10, 2014

Related U.S. Application Data
(60) Provisional application No. 61/878,133, filed on Sep. 16, 2013.

(51) Int. Cl. A61B 5/00; A61B 1/00; A61B 1/31

(52) U.S. Cl. CPC A61B 5/486; A61B 5/0084; A61B 1/31; A61B 5/4255; A61B 5/7264; A61B 1/00009
USPC 600/408

(57) ABSTRACT

Computer-implemented methods of improved quantitative interpretation of cell index profiles are provided.

### CROSS-REFERENCE TO RELATED APPLICATIONS

This application claims the benefit of U.S. Application Ser. No. 61/878,133, filed Sep. 16, 2013. The disclosure of the prior application is considered part of (and is incorporated by reference in) the disclosure of this application.

### TECHNICAL FIELD

This document generally describes improved quantitative interpretation of cell index profiles.

### BACKGROUND

Colorectal cancer (CRC) is the second leading cause of cancer deaths in the United States. A significant proportion of CRC cases can be prevented through screening with colonoscopy. For patients with average risk for CRC, the first colonoscopy is recommended at 50 years of age. The follow up interval for the subsequent colonoscopy is determined by the primary care provider by reviewing the findings of the previous colonoscopy. However, the guidelines that determine the follow-up interval are complex and the health care provider often fails to make the optimal recommendation. Consequently, 41% of patients do not receive adequate screening.

### SUMMARY

This document generally describes a guideline based clinical decision support system (CDSS) that can aid health care providers in determining and providing recommendations for colonoscopy examinations to patients. The system can automatically analyze multiple sources of clinical information in electronic health records (EHRs) and can provide guideline-based examination recommendations, including timing for first examinations and follow-up intervals.

In general, one aspect of this document features a computer-implemented method comprising, or consisting essentially of, (a) accessing, by a computer system, medical information for a patient, the medical information including, at least, a procedure note regarding a colonoscopy performed on the patient and a pathology report for the colonoscopy, the procedure note and the pathology report being in a freetext format; (b) converting, by the computer system, the procedure note and the pathology report from the freetext format to a structured output that includes additional information and concepts that are not explicitly stated in the procedure note and the pathology report; (c) identifying, based on i) the structured output of the procedure note and the pathology report and ii) other portions of the medical information for the patient, polyp information for the patient, personal and family medical history for the patient, and presence of hereditary medical conditions for the patient; (d) determining, using one or more colonoscopy guideline rules, a recommendation for a follow-up colonoscopy for the patient based, at least in part, on the polyp information for the patient, the personal and family medical history for the patient, and the presence of hereditary medical conditions for the patient; and (e) outputting the recommendation for the follow-up colonoscopy. The other portions of the medical information for the patient can include one or more of: demographic information for the patient, coded list of medical problems for the patient, colonoscopy findings recorded by a nurse, and answers provided by the patient to a questionnaire. The medical information additionally can include colonoscopy indications; and the colonoscopy indications can be additionally converted to the structured output. The polyp information for the patient can include one or more of: histology of the patient's polyps, size of the patient's polyps, number of the patient's polyps, and degree of dysplasia of the patient's polyps. The personal and family medical history for the patient can include information that identifies whether the patient or a member of the patient's family has a history of adenomatous polyps or colorectal cancer. The presence of hereditary medical conditions for the patient can include information that identifies whether the patient has colorectal cancer syndrome or inflammatory bowel disease. The recommendation for the follow-up colonoscopy can be selected from the group consisting of: gastrointestinal specialist consultation, repeat colonoscopy within a few days, follow-up colonoscopy in one year, follow-up colonoscopy in three years, follow-up colonoscopy in five years, and follow-up colonoscopy in ten years. The converting can comprise (i) pre-processing the procedure note and the pathology report to generate normalized text; (ii) mapping word patterns in the normalized text to concepts to generate mapped output; (iii) associating portions of the mapped output to higher order concepts to generate parser output; and (iv) identifying, by the computer system, particular portions of the one or more colonoscopy guideline rules to which the portions of the parser output pertain. The identifying can be based on salience weights for rules in the one or more colonoscopy guideline rules. The salience weights can indicate which findings have more significant and greater priority for determining the recommendation for the follow-up colonoscopy.

The details of one or more implementations are set forth in the accompanying drawings and the description below. Various advantages can be provided by certain implementations. For example, the described computer methods and system can provide improved standardized patient care, safety as it relates to the need for repeat screening or surveillance, coordination and enhanced integration of the practice, utilization of existing GIH and CRS resources, and the potential of significantly increasing endoscopy volume and the subsequent surgery related to neoplasia detection. By establishing more clearly-defined practice standards and management strategies, unnecessary variability in current clinical services processes and outcomes can be reduced. The disclosed computer-implemented methods and systems may also better meet the needs of the patient and referring physicians. Furthermore, the disclosed computer-implemented methods and systems can provide ongoing patient and professional education, quality improvement, and academic activities and potential transfer to other departments. This model may serve as infrastructure for other screening and surveillance modalities in the medical practice

Other features, objects, and advantages will be apparent from the description and drawings, and from the claims.

### DESCRIPTION OF DRAWINGS

FIG. 1 depicts an example computer-based CDSS.

FIG. 2 depicts example processing components of a freetext processor, example data that is processed by such components, and example processing operations that are performed by the processing components.

FIG. 3 is a flowchart that depicts an example guideline rulebase.

FIG. 4 is a flowchart that depicts an example guideline rulebase.

FIG. 5 is a block diagram of example computing devices.

FIG. 6 is a flowchart that depicts an example guideline rulebase.

FIG. 7 is a flowchart that depicts an example guideline rulebase for recurrent polyp loops.

Like reference symbols in the various drawings indicate like elements.

### DETAILED DESCRIPTION

This document describes computer-based techniques for generating and providing recommendations for colonoscopies to health care providers and/or patients.

Care providers can refer patients for colonoscopy to investigate gastrointestinal symptoms or as a colorectal cancer (CRC) screening procedure. Colonoscopy has high sensitivity for detecting CRC and precancerous adenomatous polyps and can allow for complete examination of the colon and rectum, and for the removal of polyps at the time of detection.

Upon completion of a colonoscopy procedure and after obtaining the results from pathologic examination of any biopsied tissue, the referring care provider can decide on the timing of the next colonoscopy and/or the need for further evaluation by a specialist, such as a gastroenterologist. To make a proper recommendation, the care provider can review the findings of current and/or prior colonoscopy and pathology reports to determine a multitude of parameters, such as i) histology, size, number, and/or degree of dysplasia of polyp findings, ii) the patient's risk for CRC that depends on personal and/or family history of adenomatous polyps and/or CRC, and iii) presence of conditions like hereditary CRC syndromes and/or inflammatory bowel disease.

Several health organizations have developed guidelines to determine the follow-up interval. However, the guidelines are complex and due to the multitude of parameters involved, the care provider may fail to make optimal recommendations to patients regarding colonoscopy follow-up intervals and screening. Consequently, there has been overuse of surveillance colonoscopy among low-risk subjects and underuse among high-risk subjects. Non-adherence to the recommended screening intervals can put patients at risk since each colonoscopy poses a risk for colonic perforation or GI tract hemorrhage while failure to perform colonoscopy at shorter intervals for the patients with high-risk conditions may result in cancer arising between colonoscopic exams.

To address these and other issues, a guideline based clinical decision support system (CDSS) has been developed to aid the referring care provider and others in providing colonoscopy recommendations. Such a system can automatically analyze multiple sources of relevant clinical information and provide follow-up recommendations based on one or more available guidelines.

Although clinical decision tools have been developed to support a variety of clinical decisions, support tools for colonoscopy surveillance have been limited due, at least in part, to the complexity associated with such guidelines and the large quantity of patient information that is used to make such decisions. Automatic scanning of electronic health records (EHR) to obtain patient medical information in freetext form and natural language processing (NLP) techniques have been used with limited success with regard to supporting clinical decisions due to low degrees of accuracy.

This document describes constructing and evaluating a computer system for providing colonoscopy follow-up and screening recommendations with at least a threshold degree of accuracy. In particular, this document describes using comprehensive guidelines for colonoscopy screening that address all possible patient scenarios and using NLP to accurately interpret relevant patient information in freetext reports.

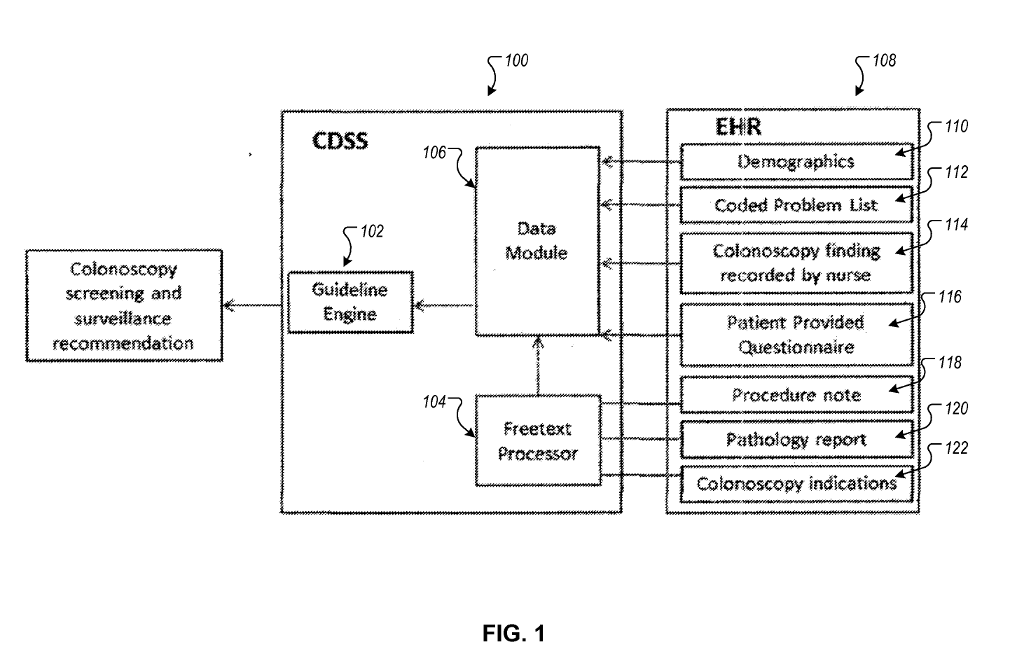

FIG. 1 depicts an example computer-based CDSS 100 with 3 modules: i) a guideline engine 102, ii) a freetext processor 104, and iii) a data module 106. The data module 106 can interface with an EHR system 108 to seek patient data and depends on the freetext processor 104 to convert freetext reports into a form that can be processed by the guideline engine 102. The guideline engine 102 can be constituted by rules and the freetext processor 104 can include rules and dictionaries, as described below.

The CDSS 100, its components (the guideline engine 102, the freetext processor 104, and/or the data module 106), and/or the EHR system 108 can be each implemented in one or more of: computer hardware (e.g., processors, memory, application specific integrated circuits (ASIC)), computer software (e.g., stored computer instructions), and firmware. Additionally, these components 100-108 may be implemented as part of the same or different computer systems, which may each include one or more computing devices, and may communicate with each other through the use of one or more networks (e.g., the internet, local area network (LAN), wide area network (WAN), virtual private network (VPN), or any combination thereof).

Guidelines used by the guideline engine 102 for colonoscopy surveillance draw upon expertise from a variety of specialties, including gastroenterologists (GE), gastrointestinal (GI) neoplasia gastroenterologists, and colorectal surgeons. The guidelines can be in the form of a flowchart, which can be implemented as set of if-then rules that provide a guideline rulebase. For a given patient, the rules can be used to initiate a lookup of particular information for the patient in the data module 106, which initiates a connection to a relevant portion of the EHR 108 and retrieves the particular information. Depending on the information that is obtained by the data module 106, the next rule in a sequence/pattern of rules for the guidelines can be evaluated according the sequence/pattern of rules (e.g., a flowchart) that are outlined in the guideline. This evaluation of rules can continue until the sequence/pattern of rules (flowchart) has been traversed in order to arrive at a recommendation. In one example sequence/pattern of rules (flowchart) that is implemented as a guideline, the guideline can include a total of 172 rules that represent 43 nodes and 88 edges that connect particular nodes to each other.

The data sources that are relevant to evaluation of the guideline rulebase are depicted as being part of the EHR 108 in FIG. 1. For example, these data sources can include one or more of the following: a) patient demographics 110, such as patient registration information, b) coded problems lists 112, such as provider managed listing in-house codes for patient problems, c) patient provided questionnaire information 116, which can include responses to questionairres administered during patient visits, such as annual check-up appointments, d) colonoscopy indications 122 based on assessments from one or more providers who examined and/or referred the patient for a colonoscopy, e) colonoscopy findings 114 recorded by a registered nurse (RN), such as during a colonoscopy in a structured format, f) procedure notes 118, such as notes transcribed from dictations made by an endoscopist after performing a colonoscopy, and g) pathology reports 120, such as specimens biopsied during a colonoscopy.

There can be a variety of freetext data sources for the CDSS 100, such as the pathology report 120, the procedure note 118, and/or the indications for colonoscopy 122. The rulebase for the freetext processor 104 can include dictionaries and a variety of processing components, such as a pre-processor, a lexer, a parser, and a post-processor.

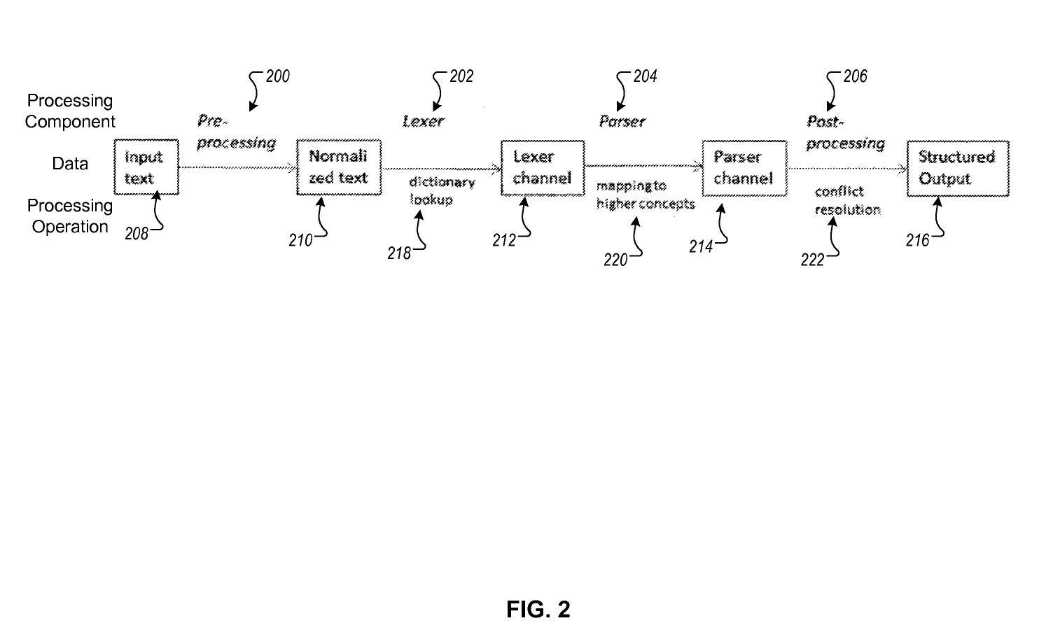

FIG. 2 depicts example processing components 200-206 of a freetext processor (e.g., the freetext processor 104), example data 208-216 that is processed by such components, and example processing operations 218-222 that are performed by the processing components. The depicted components include a pre-processor 200, a lexer 202, a parser 204, and a post-processor 206. The processing components 200-206 can be implemented across one or more computer systems.

The pre-processor 200 converts the input text 208, which can include one or more freetext data sources (e.g., procedure notes 118, pathology reports 120, colonoscopy indications 122), into a usable format for the lexer 202, such as by removing punctuation and extra spaces from the input text 208 and converting the input text 208 to lowercase. This converted text that is output by the pre-processor 200 can be normalized text 210. The lexer 202 can perform a dictionary lookup operation 218 using the normalized text 210 to map word patterns to concepts. These mapped word patterns and concepts can be loaded into a lexer channel 212 by the lexer 202. The parser 204 can search the mapped word patterns and concepts in the lexer channel 212 to map the lexer concepts to higher order concepts (220). These higher concept mappings can be output by the parser 204 into the parser channel 214. The post-processor 206 can perform a conflict resolution operation 222 to map the concepts in the parser-channel 214 to predefined parameter values that are associated with/part of a guideline rulebase. The conflict resolution 222 by the post-processor 206 can be facilitated by a variety of features, such as salience weights for rules used by the post-processor 206. Salience weights can be used to ensure that more significant findings have a higher priority for determining the output parameters. The post-processor can output the results of the conflict resolution 222 as structured output 216.

For example, if a pathology report contains the phrase “tubular Adenoma,” the preprocessor 202 can normalized the phrase to lower case form, “tubular adenoma” (example normalized text), and the lexer channel 212 is loaded with the corresponding concept ‘GTA’ by the lexer 202, which leads to the parser 204 using rules to load higher order concept of ‘PT_AdenomatousT’ (corresponding to group 1 in an example guideline flowchart) in the parser channel 214. The post-processor 206 uses rules to ensure that this output is retained even if other types of polyps are noted in the report, as the finding of group 1 is weighted higher than findings of other polyp types.

Text processors (e.g., freetext processor 103) can use different sets of dictionaries for each of the different freetext data sources, such as the procedure note 118, the pathology report 120, and the colonoscopy indications 122. The dictionaries can be constructed by analysis of corpora for each of the data sources. For instance, pathology reports, procedure notes, and indication entries can be analyzed to identify word patterns specific to the corpora type and to identify rules to map the word patterns to parameter values of interest.

Additionally, input from experts (e.g., pathologists and physicians) can be used to identify implicit knowledge that can be used for interpreting the text documents and to create rules to represent the implicit knowledge. For example, if a pathologist does not mention a finding of cancer, the referring physician can interpret that there was no finding of cancer (example of implicit knowledge). Also, if the pathologist mentions findings of tubular adenoma and hyperplastic adenoma, the referring physician may consider the finding of the tubular adenoma to be more significant and may base further decisions on this finding. Such implicit knowledge can be identified from practitioner input and used to create rules for interpreting textual documents.

Performance of the freetext processor 104 can be adjusted and verified using word patterns identified in the corpus analysis and using random sets of pathology reports, procedure notes, and/or indication entries.

An implementation of the CDSS 100 was evaluated on a test set of 53 patients, which was constructed as follows. This implementation of the CDSS 100 was applied to a set of randomly selected 1000 patients who had a colonoscopic biopsy. The patients were grouped on the basis of pathways that were traversed in an example guideline flowchart. Each pathway in the flowchart can correspond to a unique set of patient parameters and can represent a unique decision scenario.

The test set was generated by randomly selecting cases for each of the decision scenarios, such that a maximum of 5 patients were selected for a particular decision scenario. Such a weighted random selection was used to reduce the evaluation bias towards frequently occurring decision scenarios. In 131 of the 1000 cases, the example implementation of the CDSS 100 indicated that there was insufficient or vague documentation in an example implementation of the EHR 108 to compute the recommendation, and these cases were excluded for creation of the test set.

For each of the 53 cases in the test set, the recommendation made by the example implementation of the CDSS 100 was evaluated as follows. For each case, a GE was blinded to the recommendation of the example implementation of the CDSS 100 and was requested to make an initial recommendation. When there was a mismatch in the recommendation of the GE and the example implementation of the CDSS 100, the GE was unblinded to output of the example implementation of the CDSS 100 and was requested to reconsider revision of his/her initial recommendation. An error analysis was performed by comparing the logic steps for the recommendations.

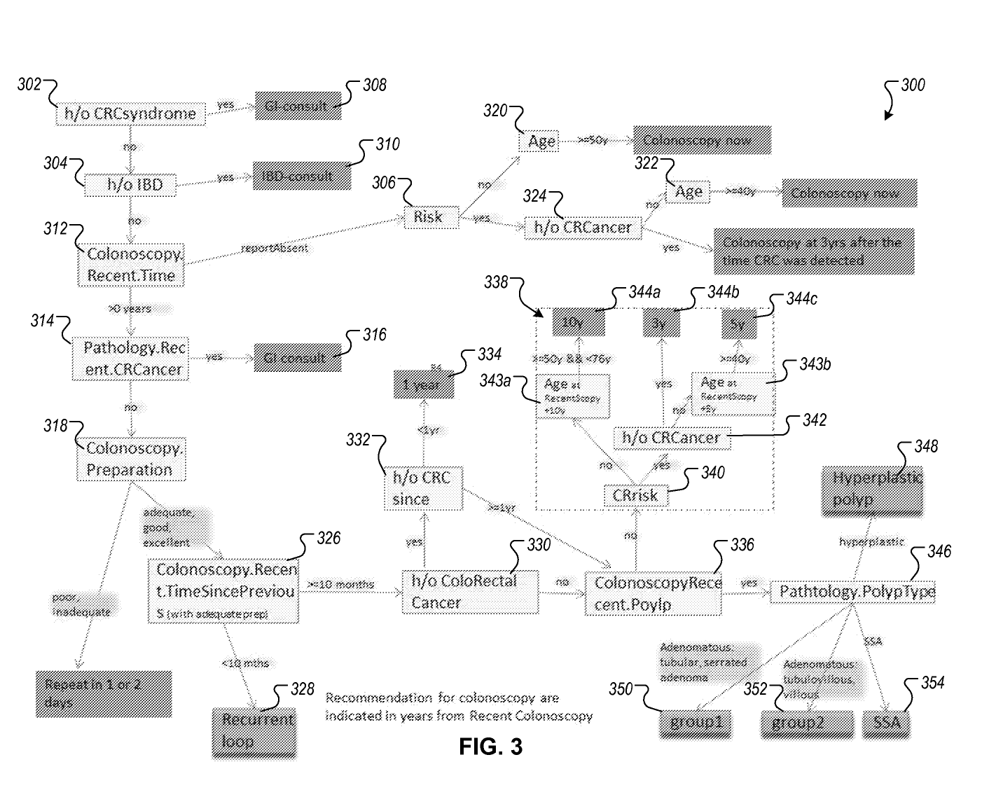

FIG. 3 is a flowchart that depicts an example guideline rulebase 300. The example guideline rulebase 300 can be processed by any of a variety of appropriate computing devices, such as the CDSS 100 and/or the guideline engine 102 of the CDSS 100. The example guideline rulebase 300 is depicted as including the following elements: risk status, CDC syndrome, inflammatory bowel disease (IBD), polyp, and preparation (prep).

Risk status: A patient is high risk for CRC, if there is any personal history of CRC or adenomatous polyps, or if there is any family history of CRC.

CRC syndrome: This term includes conditions like Lynch syndrome, familial adenomatous polyposis, Peutz-Jeghers syndrome, etc.

Inflammatory bowel disease (IBD): This includes chronic ulcerative colitis (CUC), Crohn's disease, indeterminant IBD or colitis.

Polyp: Histological features including type and degree of dysplasia can be determined from pathology reports. The size, number, and side (location) of polyp can be determined from analysis of the finding recorded in the GI database. The number of polyp findings can be added up to compute the number of polyp. The diameter of largest polyp reported can be considered as the polyp size. Polyp side can be left, right, or both, and can be determined from anatomic locations recorded for the polyp findings. Information about whether the polyp was removed completely, piecemeal, or incompletely can be determined from procedure notes.

Preparation (prep): This indicates whether the colon could be fully visualized without any obstruction by stool. Patients prepare for the colonoscopy with minimum of a 24 hour liquid only diet and a bowel cleanse with polyethylene glycol solution or other cathartic agent.

A brief overview of the flowchart depicted in FIG. 3 is as follows. The parameters/concepts that are discussed in the flowchart depicted in FIG. 3 are derived from the data sources that are outlined in Table 1 below.

TABLE 1

| Parameter/Concept | Data Source |
|---|---|
| Age | Demographics |
| CRC risk | Problem list, colonoscopy |
| | indication* and patient provided |
| | information |
| history of CRC syndrome | Problem list, colonoscopy |
| | indications* |
| history of IBD | Problem list, colonoscopy |
| | indications* |
| patient's preparation for | Data recorded by nurse, procedure |
| colonoscopy | note* |
| polyp histology: type and degree | Pathology report* |
| of dysplasia, or if cancer is | |
| present | |
| polyp gross characteristics: | Colonoscopy findings |
| number, size, and side | |
| polyp removal | Procedure note* |

*Freetext data source

The guideline engine can begin by determining whether the patient has CRC syndrome (302) and IBD (304), and whether the patient is at risk for CRC (306), by checking the problem list and colonoscopy indications. The patient responses to related questionnaire are additionally analyzed to determine risk status. If the patient has CRC syndrome (302) or IBD (304), a referral is suggested to the gastroenterology (GI) neoplasia clinic (308) or for IBD consultation (310), respectively. If the recent pathology report (312) mentions a cancer finding (314), the patient is referred to the GI neoplasia clinic (316).

Next, the colonoscopy findings are looked up (318). If there is no record of any prior colonoscopy procedure (312), the patient's age (320, 322), risk status (306), and previous history of CRC (324) are considered to recommend the first colonoscopy date. Next, the patients who have recent recurrent polyp identified by less than 10 months in their last two colonoscopies (326) are filtered out (328), as they are not addressed in the technique outlined in the flowchart depicted in FIG. 3. Patients with history of CRC (330) diagnosed in the last year (332) are recommended for colonoscopy one year after diagnosis (334). A determination is made as to whether polyps were found in recent colonoscopies (336). For the majority of patients no polyp is found in the recent colonoscopies (338), and the patients' risk (340), history of CRC (342), and age (343a-b) are considered for recommending the follow up interval (344a-c). If a polyp has been biopsied in the last colonoscopy, the histology is interpreted and mapped (346) to one of the following four categories: hyperplastic (348), group 1 (tubular adenoma, serrated adenoma) (350), group 2 (tubulovillous adenoma, villous adenoma) (352), and sessile serrated adenoma (354). These groups are outlined below in Table 2. For each of the four categories of the polyps, the degree of dysplasia, size, number, and the removal condition of the polyp are considered to determine the follow up interval.

TABLE 2

| Freetext | | |
|---|---|---|
| source | Parameter | Possible values |
| Pathology | type of polyp | Hyperplastic, sessile serrated adenoma, |
| report | | group 1(tubular adenoma, serrated |
| | | adenoma), group 2 (tubulovillous |
| | | adenoma, villous adenoma), or other |
| | degree of polyp | no, indefinite, low or high |
| | dysplasia | |
| | cancer | present or absent |
| Procedure | removal of the | complete, incomplete, or piecemeal |
| note | polyp | |
| | preparation | Adequate or inadequate |
| Colonoscopy | personal history | yes or no |
| indications | of polyps | |
| | personal history | yes or no |
| | of CRC | |
| | personal history | yes or no |
| | of IBD | |
| | family history | yes or no |
| | of CRC | |

Components of a freetext processor (e.g., freetext processor 104) for the different report types are described below. Table 2 provides a mapping of the report type to the structured parameters that can be output by a freetext processor. As complex concepts can be extracted from pathology reports, the text processor can use a rulebase to process the logic steps to properly interpret for pathology report. On the other hand, interpreting procedure notes and colonoscopy may be possible with only a pre-processing step (200), dictionary lookup (218), and minimal conflict resolution as the extracted concepts can be simple. Such an approach to constructing a report specific dictionary can differ from the approaches of other clinical natural language processing applications, which have used a single dictionary to identify concepts in different types of reports.

The nomenclature of pathology reports may have been standardized at the institution. The reports can consist of a gross description of a specimen, followed by the histology. A standard static template of follow-up recommendations can be found in the end of gastroenterology pathology reports. The corpus analysis can reveal that the histology section of the report uses a standard vocabulary and template, to report the type of polyp, degree of dysplasia, and finding of cancer. A freetext processor can focus on the histology section of such pathology reports. The preprocessor step (200) can identify the histology section by using regular expressions for section headings. The lexer (202) and parser (204) can use rules to identify word patterns and map them to higher level concepts, respectively. The post-processor (206) can use weighted rules to establish the following precedence order for polyp finding: ssa, group2, group1, and hyperplastic. Such an order can ensure that the finding of sessile serrated adenorna (ssa) is weighted higher than the finding of hyperplastic polyp, so that the appropriate guideline can be applied.

Procedure notes can be transcribed from post-procedure dictation recorded by a practitioner (e.g., an endoscopist) and, based on the procedure notes being derived from transcribed dictation, there may be less grammatical structure. The freetext processor can perform a dictionary lookup operation to identify word patterns for inadequate bowel preparation, piecemeal removal of polyps, and polyp size, as indicated below in Table 3. The polyp size annotations can further be analyzed to determine the size of the largest polyp.

TABLE 3

| Concept | Word patterns |
|---|---|
| inadequate | limited visualization |
| prep | suboptimal visual |
| | suboptimal bowel prepartion |
| | suboptimal preparation |
| | suboptimal prep |
| | poorly prepped |
| piecemeal | Piecemeal |
| removal | |
| polyp size | \d+\.*\d* [m\|c]m (\w+)*polyp\w* |
| | \d+\.*\d* [m\|c]m x \d+\.*\d* [m\|c]m |
| | (\w+)*polyp\w* |
| | \d+\.*\d* [m\|c]m x \d+\.*\d* [m\|c]m x |
| | \d+\.*\d* [m\|c]m (\w+)*polyp\w* |

Referral indications for procedures noted by a referring physician can be mapped with a dictionary lookup to word patterns for IBD, personal/family history of polyp/colorectal cancer. Table 4 shows word patterns for some of the concepts.

TABLE 4

| Concept | Word patterns |
|---|---|
| ibd | crohn s colitis |
| | colitis ulcerative |
| | ulcerative proctitis |
| | unexplained diarrhea ibd initial diagnosis |
| p/h/o | [\|family][\w\|\s] + colorectal cancer |
| colorectal | [\|family][\w\|\s] + colon ca |
| cancer | [\|family][\w\|\s] + rectal ca |
| | [\|family][\w\|\s] + hx colon ca |
| | [\|family][\w\|\s] + ca colon |
| | [\|family][\w\|\s] + polyp cancer |

During construction of an example test set for manual evaluation from a larger set of 1000 patients, the CDSS indicated that 131 (13%) patients documentation for polyp findings in the EHR was missing or incomplete. Only half the example set of patients undergoing colonoscopy had a biopsy, a hypothesis was generated that the CDSS would label less than 7% of the patients for missing information, which may be an acceptable rate. The CDSS may be unable to generate a recommendation for the referring care provider in such cases (missing information).

The example test set contained cases for 28 different decision scenarios. The frequency distribution of the case scenarios in this example set is depicted below in Table 5, which indicates that the test set included a wide variety of decision scenarios, which facilitated a comprehensive evaluation of the CDSS.

TABLE 5

| Number | CRC | | | | | Prep | CRC | | | | Polyp | Polyp | Dys- | | |
|---|---|---|---|---|---|---|---|---|---|---|---|---|---|---|---|
| of | Syn- | | Report | Specimen | Recent | Inade- | within | CRC | Polyp | Polyp | Size | Num- | pla- | Recom- | |
| cases | drome | IBD | absent | collected | Cancer | quate | 1 year | Risk | Type | Side | (cms) | ber | sia | mendation | |
| 2 | | | | | | | | Y | group1 | | <1 | <3 | low | 5 | years |
| 2 | | | | | | | | Y | group1 | | <1 | <3 | low | 3 | years |
| 2 | | | | | | | | | group1 | | | | high | 2-3 | months |
| 1 | | | | | | | | | group1 | | <1 | 3-9 | low | 3 | years |
| 1 | | | | | | | | | group1 | | ≧2 | | low | 2-3 | months |
| 1 | | | | | | | | | group1 | | ≧1 & | | low | 2-3 | months |
| | | | | | | | | | | | <2 | | | | |
| 3 | | | | | | | | | group2 | | ≧2 | ≧2 | low | 2-3 | months |
| 2 | | | | | | | | | group2 | | ≧1 & | | | 1 | year |
| | | | | | | | | | | | <2 | | | | |
| 1 | | | | | | | | | group2 | | <1 | <3 | low | 1 | year |
| 1 | | | | | | | | | group2 | | ≧1 & | ≧1 & | low | 2-3 | months |
| | | | | | | | | | | | <2 | <2 | | | |
| 3 | | | | | | | | | hyper- | left | <1 | <30 | | 5 | years |
| | | | | | | | | | plastic | | | | | | |
| 1 | | | | | | | | Y | hyper- | left | <1 | <30 | | 5 | years |
| | | | | | | | | | plastic | | | | | | |
| 1 | | | | | | | | | hyper- | right/ | <1 | <3 | | 10 | years |
| | | | | | | | | | plastic | both | | | | | |
| 1 | | | | | | | | Y | hyper- | | <1 | <30 | | 3 | years |
| | | | | | | | | | plastic | | | | | | |
| 1 | | | | | | | | Y | hyper- | | <1 | <3 | | 3 | years |
| | | | | | | | | | plastic | | | | | | |
| 3 | | | | | | | | | SSA | | | ≧3 | | GI-consult | |
| 2 | | | | | | | | | SSA | | | <3 | | 3 | years |
| 5 | | Y | | | | | | | | | | | | IBD-consult | |
| 5 | | | | | | | | | | | | | | 10 | years |
| 5 | | | | Y | | | | | | | | | | 10 | years |
| 2 | | | Y | | | | | | | | | | | Now | |
| 2 | | | | | Y | | | | | | | | | GI-consult | |
| 1 | | | | | | Y | | | | | | | | 1/2 | days |
| 1 | | | | | | | | Y | | | | | | 5 | years |
| 1 | | | | | | | | | | | | | | 10 | years |
| 1 | Y | | | | | | | | | | | | | GI-consult | |
| 1 | | | | | | | Y | | | | | | | 1 | year |

| | |
|---|---|
| 1 | NIL |

Empty cells indicate that the parameter value was negative or not considered in the decision scenario.
Y indicates that the value of the parameter was true.

The evaluation on the test set revealed that in 45 of the 53 test cases the recommendation of the CDSS matched with the initial recommendation of the gastroenterologist (GE). In 5 cases the GE retained her initial recommendation, as the CDSS recommendation was not optimal and in three test cases the CDSS helped the GE to revise her initial blinded recommendations.

The 5 cases for which the CDSS failed to generate the optimal recommendation were further analyzed, as depicted in Table 6 below. In two of these test cases the CDSS had failed to correctly determine CRC risk and history of IBD. These errors could be resolved by extending the list of conditions searched for determination of CRC risk and history of IBD, and by extending the dictionaries in the text-processor. Although the tested list had been generated to be comprehensive, the test list missed some conditions.

TABLE 6

| CDSS | GE | |
|---|---|---|
| recommendation | recommendation | Reaston for CDSS error |
| 1 year | 2-3 months | largest polyp not noted by RN |
| 10 years | GI-consult | polyp finding not noted by RN |
| 1 year | GI consult | largest polyp not noted by RN |
| 10 year | 3 years | CDSS missed h/o CRC |
| 5 years | IBD-consult | CDSS missed IBD condition |

The time intervals in the recommendations are for the next colonoscopy.

In the other three test cases where the CDSS failed to generate the optimal recommendation, the polyp size was not noted or was noted incorrectly in the findings database abstracted by the RN. In two cases the largest polyp was not documented. Modifications to the CDSS resolved the errors resulting from these test cases.

In the three test cases in which the CDSS helped the GE to revise her initial blinded recommendations, the revision in the GE's recommendations were mainly due to difficulty in recalling exact numerical cut-offs for polyp size/number parameters in the guideline flowchart. The GE participating in the test evaluation is an expert on the colonoscopy surveillance and screening guidelines. The CDSS can be used by referring care providers, including nurse practitioners, physician assistants, primary care physicians, or other specialists. The referring care providers may not usually be familiar with recommendation guidelines and, accordingly, the CDSS can be a useful resource to provide expert guidance for navigating the multitude of factors for consideration when generating a recommendation.

A variety of challenges were overcome and resulted in the development of the CDSS. For example, one challenge to development of CDSS has been the lack of consensus guidelines in practice. Previously, tools that have been routinely used today have been limited to narrow decision scopes where guidelines are well established. For instance, reminders for abnormal lab tests, drug interactions, and immunization scheduling have been shown to be used widely and effectively. The lack of a consensus on guidelines for more complex decision problems like colonoscopy screening/surveillance, has possibly delayed the development of a CDSS in this area in the past. Another hurdle for CDSS development has been the challenge of extracting patient information from freetext reports. These obstacles were overcome by developing a comprehensive decision flowchart based on high quality consensus guidelines and by using natural language processing methods to utilize freetextdata.

Natural language processing of freetext information (e.g., procedure notes, pathology reports, colonoscopy indications) was aided by the use of templates for the various freetext information. The accuracy with which the freetext information was interpreted was improved by using complementary data sources. For instance, the lack of prep and polyp number information from the freetext procedure notes is in many cases supplemented by the RNs structured data. Similarly, the patient history used for determining presence of IBD, CRC syndrome, and CRC risk/history was found to be inadequately covered by the ER's coded problem list. The problem list was supplemented by text processing of the colonoscopy indications noted by the referring car physician.

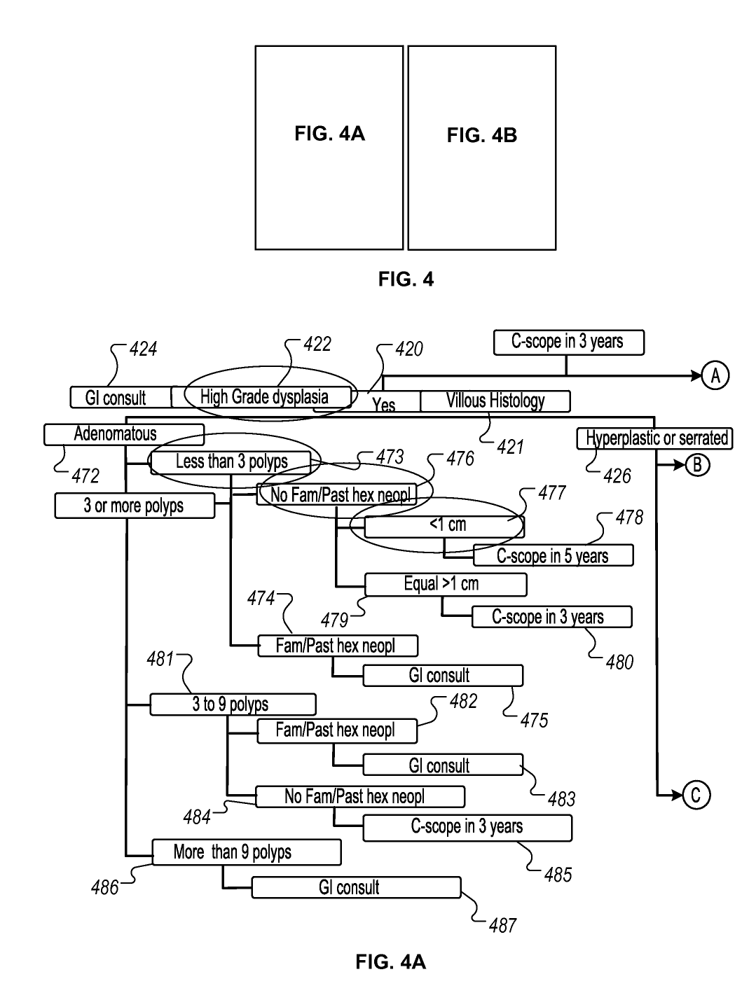

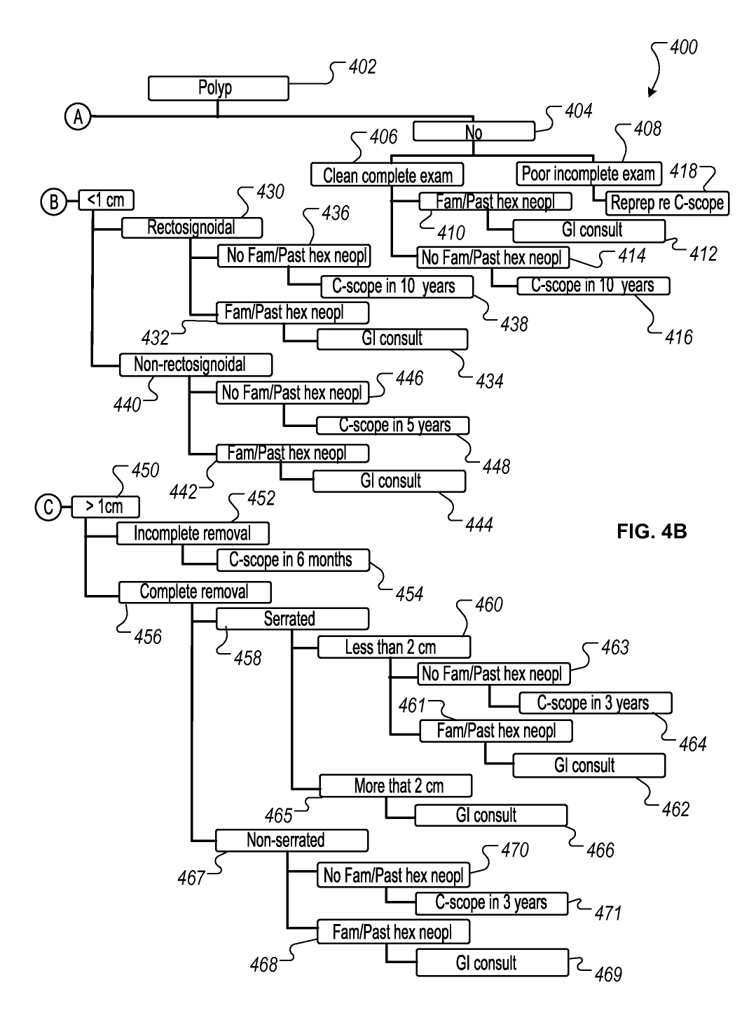

FIG. 4 is a flowchart that depicts an example guideline rulebase 400. The example guideline rulebase 400 can be processed by any of a variety of appropriate computing devices, such as the CDSS 100 and/or the guideline engine 102 of the CDSS 100. The example guideline rulebase 400 can be used alone or in combination with other guideline rulebases, such as the guideline rulebase 300. The guideline rulebase 400 takes into consideration multiple clinical factors including polyp histology, degree of dysplasia, size, location, the completeness of the prep, and past medical and family history of colon polyps or cancer.

A determination is made as to whether a polyp was detected in a colonoscopy that was performed within the past three years (402). If a polyp was not detected (404), then a determination is made as to whether the previous examination was clean and complete (406) or poor and incomplete (408). If the exam was clean and complete (406), then a determination is made as to whether the patient has a family or past personal medical history of neoplasia. If the patient has a family or past personal medical history of neoplasia (410), then a GI consult is recommended (412). If the patient does not have a family or past personal medical history of neoplasia (414), then a follow-up recommendation of 10 years for a next colonoscopy is provided to the patient (416). If the exam was poor and incomplete (408), then recommendation to repeat the colonoscopy is provided to the patient (418).

If a polyp was detected (420), an examination of the villous histology of the polyp if performed (421). If the polyp is determined to have a high grade of dysplasia (422), then a GI consult is recommended to the patient (424). If the polyp is determined to be hyperplastic or serrated (426), then the size of the polyp is examined. If the polyp is less than 1 cm in size (428), a determination as to whether the polyp is rectosigmoidal. If the polyp is rectosigmoidal (430), then a determination is made as to whether the patient has a family or past personal medical history of neoplasia. If the patient has a family or past personal medical history of neoplasia (432), then a GI consult is recommended (434). If the patient does not have a family or past personal medical history of neoplasia (436), then a follow-up recommendation of 10 years for a next colonoscopy is provided to the patient (438). If the polyp is not rectosigmoidal (440), then a determination is made as to whether the patient has a family or past personal medical history of neoplasia. If the patient has a family or past personal medical history of neoplasia (442), then a GI consult is recommended (444). If the patient does not have a family or past personal medical history of neoplasia (446), then a follow-up recommendation of 5 years for a next colonoscopy is provided to the patient (448).

If the polyp has a size of 1 cm or greater (450), then a determination is made as to whether the polyp was completely removed. If the polyp was not completely removed (incomplete removal) (452), then a follow-up colonoscopy 6-months later is recommended to the patient (454). If the polyp was completely removed (456), then a determination is made as to whether the polyp was serrated. If the polyp was serrated (458), then a determination is made as to whether the polyp was greater than or less than 2 cm in size. If the polyp was less than 2 cm in size (460), then a determination is made as to whether the patient has a family or past personal medical history of neoplasia. If the patient has a family or past personal medical history of neoplasia (461), then a GI consult is recommended (462). If the patient does not have a family or past personal medical history of neoplasia (463), then a follow-up recommendation of 3 years for a next colonoscopy is provided to the patient (464). If the polyp was greater than or equal to 2 cm in size (465), then a GI consult is recommended (466). If the polyp was not serrated (467), then a determination is made as to whether the patient has a family or past personal medical history of neoplasia. If the patient has a family or past personal medical history of neoplasia (468), then a GI consult is recommended (469). If the patient does not have a family or past personal medical history of neoplasia (470), then a follow-up recommendation of 3 years for a next colonoscopy is provided to the patient (471).

If the polyp finding is determined to be adenomatous (472), then an evaluation of the number of polyps that have been detected is performed. If less than 3 polyps were detected (473), then a determination is made as to whether the patient has a family or past personal medical history of neoplasia. If the patient has a family or past personal medical history of neoplasia (474), then a GI consult is recommended (475). If the patient does not have a family or past personal medical history of neoplasia (476), then the size of the polyp is evaluated. If the poly is less than 1 cm (477), then a follow-up recommendation of 5 years for a next colonoscopy is provided to the patient (478). If the poly is greater than or equal to 1 cm (479), then a follow-up recommendation of 3 years for a next colonoscopy is provided to the patient (480). If 3-9 polyps were detected (481), then a determination is made as to whether the patient has a family or past personal medical history of neoplasia. If the patient has a family or past personal medical history of neoplasia (482), then a GI consult is recommended (483). If the patient does not have a family or past personal medical history of neoplasia (484), then a follow-up recommendation of 3 years for a next colonoscopy is provided to the patient (485). If more than 9 polyps were detected (486), then a GI consult is recommended to the patient (487).

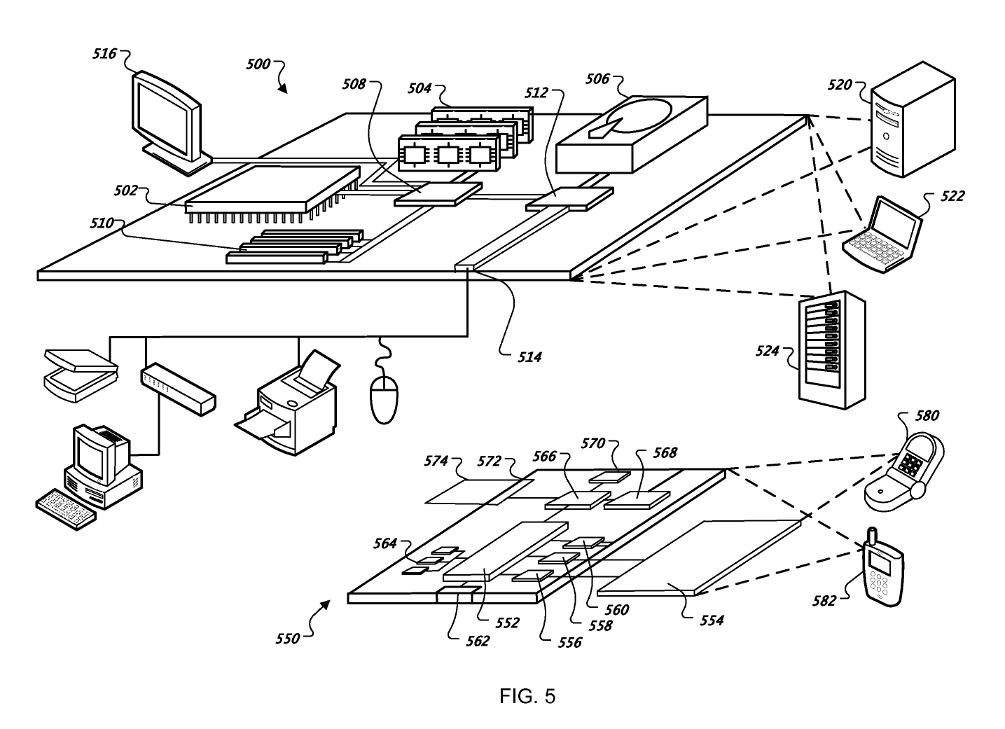

FIG. 5 is a block diagram of computing devices 500, 550 that may be used to implement the systems and methods described in this document, as either a client or as a server or plurality of servers. Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally computing device 500 or 550 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses may be used, as appropriate, along with multiple memories and types of memory. Also, multiple computing devices 500 may be connected, with each device providing portions of the necessary operations (e.g., as a server bank, a group of blade servers, or a multi-processor system).

The memory 504 stores information within the computing device 500. In one implementation, the memory 504 is a volatile memory unit or units. In another implementation, the memory 504 is a non-volatile memory unit or units. The memory 504 may also be another form of computer-readable medium, such as a magnetic or optical disk.

The storage device 506 is capable of providing mass storage for the computing device 500. In one implementation, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth-intensive operations. Such allocation of functions is exemplary only. In one implementation, the high-speed controller 508 is coupled to memory 504, display 516 (e.g., through a graphics processor or accelerator), and to high-speed expansion ports 510, which may accept various expansion cards (not shown). In the implementation, low-speed controller 512 is coupled to storage device 506 and low-speed expansion port 514. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. It may also be implemented as part of a rack server system 524. In addition, it may be implemented in a personal computer such as a laptop computer 522. Alternatively, components from computing device 500 may be combined with other components in a mobile device (not shown), such as device 550. Each of such devices may contain one or more of computing device 500, 550, and an entire system may be made up of multiple computing devices 500, 550 communicating with each other.

Computing device 550 includes a processor 552, memory 564, an input/output device such as a display 554, a communication interface 566, and a transceiver 568, among other components. The device 550 may also be provided with a storage device, such as a microdrive or other device, to provide additional storage. Each of the components 550, 552, 564, 554, 566, and 568, are interconnected using various buses, and several of the components may be mounted on a common motherboard or in other manners as appropriate.

The processor 552 can execute instructions within the computing device 550, including instructions stored in the memory 564. The processor may be implemented as a chipset of chips that include separate and multiple analog and digital processors. Additionally, the processor may be implemented using any of a number of architectures. For example, the processor 552 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 550, such as control of user interfaces, applications run by device 550, and wireless communication by device 550.

Processor 552 may communicate with a user through control interface 558 and display interface 556 coupled to a display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may be provide in communication with processor 552, so as to enable near area communication of device 550 with other devices. External interface 562 may provide, for example, for wired communication in some implementations, or for wireless communication in other implementations, and multiple interfaces may also be used.

The memory 564 stores information within the computing device 550. The memory 564 can be implemented as one or more of a computer-readable medium or media, a volatile memory unit or units, or a non-volatile memory unit or units. Expansion memory 574 may also be provided and connected to device 550 through expansion interface 572, which may include, for example, a SIMM (Single In Line Memory Module) card interface. Such expansion memory 574 may provide extra storage space for device 550, or may also store applications or other information for device 550. Specifically, expansion memory 574 may include instructions to carry out or supplement the processes described above, and may include secure information also. Thus, for example, expansion memory 574 may be provide as a security module for device 550, and may be programmed with instructions that permit secure use of device 550. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that may be received, for example, over transceiver 568 or external interface 562.

Device 550 may communicate wirelessly through communication interface 566, which may include digital signal processing circuitry where necessary. Communication interface 566 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 568. In addition, short-range communication may occur, such as using a Bluetooth, WiFi, or other such transceiver (not shown). In addition, GPS (Global Positioning System) receiver module 570 may provide additional navigation- and location-related wireless data to device 550, which may be used as appropriate by applications running on device 550.

Device 550 may also communicate audibly using audio codec 560, which may receive spoken information from a user and convert it to usable digital information. Audio codec 560 may likewise generate audible sound for a user, such as through a speaker, e.g., in a handset of device 550. Such sound may include sound from voice telephone calls, may include recorded sound (e.g., voice messages, music files, etc.) and may also include sound generated by applications operating on device 550.

The computing device 550 may be implemented in a number of different forms, as shown in the figure. For example, it may be implemented as a cellular telephone 580. It may also be implemented as part of a smartphone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

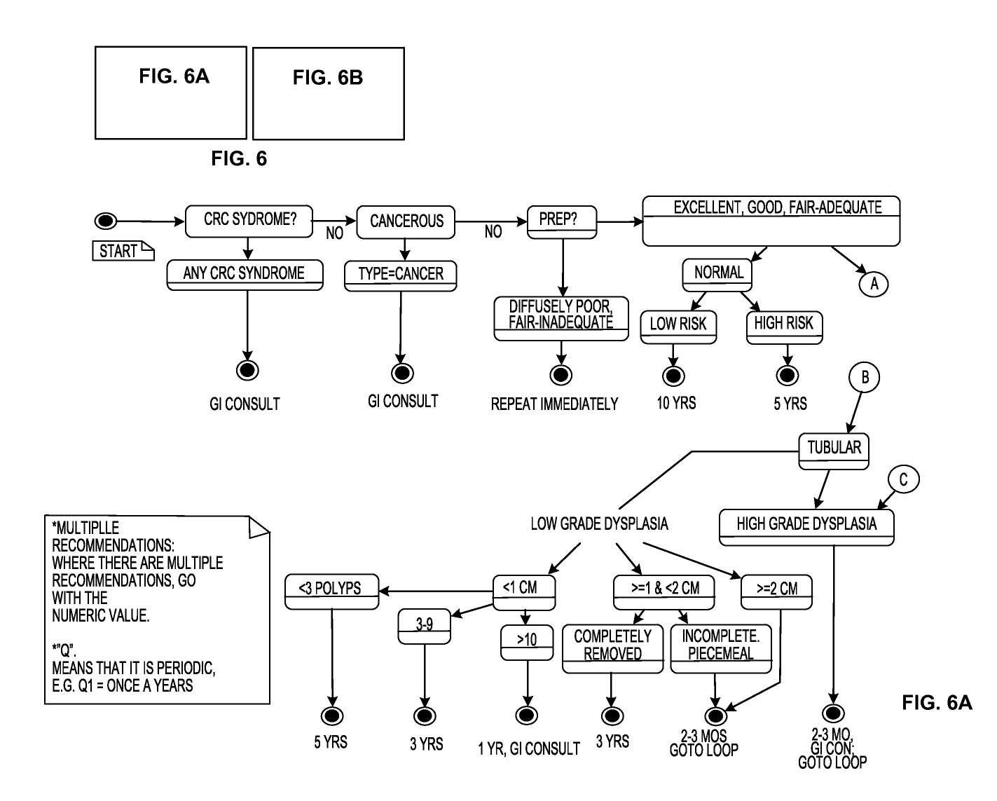

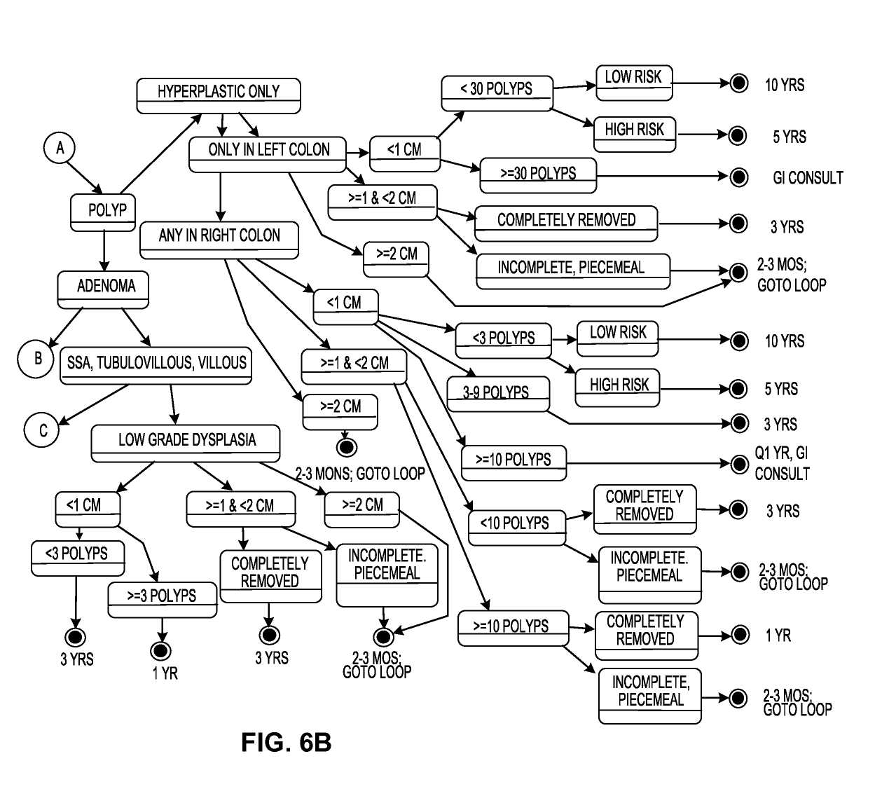

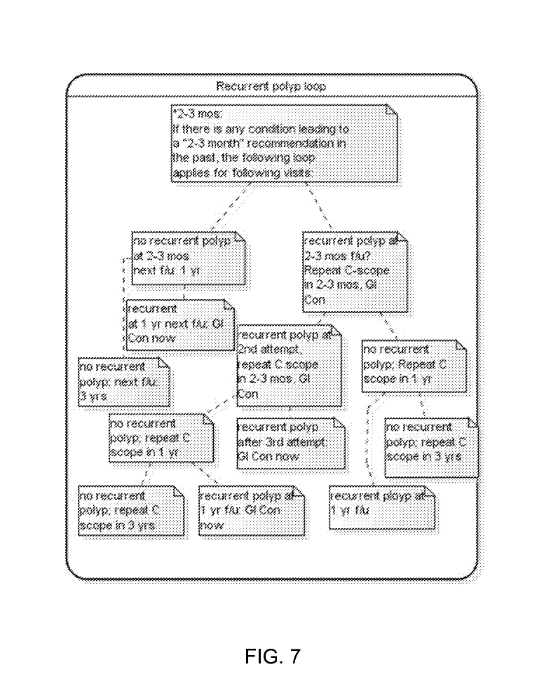

FIG. 6 is a flowchart that depicts an example guideline rulebase. The example rulebase that is depicted in FIG. 6 is similar to the guideline rulebase discussed above with regard to FIG. 4. The flowchart starts at the circle in the upper left corner next to the word “start” and, after following appropriate decision paths, terminates in colonoscopy follow-up recommendations that are identified by the circle icons containing additional circles within themselves. For example, the recommendation if there is any CRC syndrome is for a “GI consult.” For the recommendations that include the phrase “goto loop,” that indicates that the recurrent polyp loops flowchart that is depicted in FIG. 7 and described below should additionally be used to provide a recommendation.

FIG. 7 is a flowchart that depicts an example guideline rulebase for recurrent polyp loops. The recurrent polyp loop is performed for the results of a follow-up examination following a 2-3 month recommendation from the flowchart depicted in FIG. 6.

Following the left branch of the recurrent polyp loop flowchart, if no recurrent polyp is detected at the 2-3 month follow-up appointment, then the next follow-up appointment is recommended for 1 year later. If at the 1 year follow-up appointment a recurrent polyp loop is identified, then a GI consultation is recommended now (immediately). If not current polyp is detected at the 1 year follow-up appointment, then the next follow-up appointment is recommended for 3 years later.

Following the right branch of the recurrent polyp loop flowchart, if a recurrent polyp is detected at the 2-3 month follow-up appointment, then a repeat of the c-scope is recommended in 2-3 months along with a GI consultation. If a recurrent polyp is identified as part of this second attempt c-scope, then a repeat of the c-scope is recommended in 2-3 months along with another GI consult. If, after this third attempt c-scope is performed, there is still a recurrent polyp, then a GI consult is recommended not (immediately). If, instead, the third attempt c-scope does not result in detection of a recurrent poly, a repeat of the c-scope is recommended for 1 year later. If no recurrent poly is identified in this 1 year follow-up, then a repeat of the c-scope is recommended for 3 years later. If, in contrast, a recurrent poly is identified in this 1 year follow-up, then a GI consult is recommended for now.

Move back up toward the top of the tree, if no recurrent poly is detected in the second attempt GI consult, then a repeat of the c-scope is recommended for 1 year later. If no recurrent polyp is detected at this appointment, then a recommendation is provided to repeat the c-scope in 3 years. In contrast, if a recurrent poly is detected at the second attempt appointment, then a recurrent poly examination is recommended for a 1 year follow-up.

Although a few implementations have been described in detail above, other modifications are possible. Moreover, other mechanisms for determining colonoscopy follow-up recommendations may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A computer-implemented method comprising:
accessing, by a computer system, medical information for a patient, the medical information including, at least, a procedure note regarding a colonoscopy performed on the patient and a pathology report for the colonoscopy, the procedure note and the pathology report being in a freetext format;
converting, by the computer system, the procedure note and the pathology report from the freetext format to a structured output that includes additional information and concepts that are not explicitly stated in the procedure note and the pathology report;
identifying, based on i) the structured output of the procedure note and the pathology report and ii) other portions of the medical information for the patient, polyp information for the patient, personal and family medical history for the patient, and presence of hereditary medical conditions for the patient;
determining, using one or more colonoscopy guideline rules, a recommendation for a follow-up colonoscopy for the patient based, at least in part, on the polyp information for the patient, the personal and family medical history for the patient, and the presence of hereditary medical conditions for the patient; and
outputting the recommendation for the follow-up colonoscopy.

2. The computer-implemented method of claim 1, wherein the other portions of the medical information for the patient include one or more of: demographic information for the patient, coded list of medical problems for the patient, colonoscopy findings recorded by a nurse, and answers provided by the patient to a questionnaire.

3. The computer-implemented method of claim 1, wherein the medical information additionally includes colonoscopy indications; and
wherein the colonoscopy indications are additionally converted to the structured output.

4. The computer-implemented method of claim 1, wherein the polyp information for the patient includes one or more of: histology of the patient's polyps, size of the patient's polyps, number of the patient's polyps, and degree of dysplasia of the patient's polyps.

5. The computer-implemented method of claim 1, wherein the personal and family medical history for the patient includes information that identifies whether the patient or a member of the patient's family has a history of adenomatous polyps or colorectal cancer.

6. The computer-implemented method of claim 1, wherein the presence of hereditary medical conditions for the patient includes information that identifies whether the patient has colorectal cancer syndrome or inflammatory bowel disease.

7. The computer-implemented method of claim 1, wherein the recommendation for the follow-up colonoscopy is selected from the group consisting of:
gastrointestinal specialist consultation, repeat colonoscopy within a few days, follow-up colonoscopy in one year, follow-up colonoscopy in three years, follow-up colonoscopy in five years, and follow-up colonoscopy in ten years.

8. The computer-implemented method of claim 1, wherein the converting comprises:
pre-processing the procedure note and the pathology report to generate normalized text;
mapping word patterns in the normalized text to concepts to generate mapped output;
associating portions of the mapped output to higher order concepts to generate parser output; and
identifying, by the computer system, particular portions of the one or more colonoscopy guideline rules to which the portions of the parser output pertain.

9. The computer-implemented method of claim 8, wherein the identifying is based on salience weights for rules in the one or more colonoscopy guideline rules.

10. The computer-implemented method of claim 9, wherein the salience weights indicate which findings have more significant and greater priority for determining the recommendation for the follow-up colonoscopy.
